(19) United States
(12) Patent Application Publication
(10) Pub. No.: US 2023/0067989 A1
(43) Pub. Date: Mar. 2, 2023

(54) WALL-MOUNTED AIR PURIFIER

(71) Applicant: SHENZHEN ANTOP TECHNOLOGY CO., LTD, Shenzhen (CN)

(72) Inventor: Ruidian YANG, Shenzhen (CN)

(21) Appl. No.: 17/578,002

(22) Filed: Jan. 18, 2022

(30) Foreign Application Priority Data
Aug. 30, 2021 (CN) 202122073830.5

(51) Int. Cl. A61L 9/18; B01D 46/00; B01D 53/00; B01D 53/88

(52) U.S. Cl. CPC A61L 9/18; B01D 46/0028; B01D 53/007; B01D 53/885; B01D 2279/65; B01D 2279/50; B01D 2257/91; B01D 2255/802; A61L 2209/14; A61L 2209/12

(57) ABSTRACT

The present application provides a wall-mounted air purifier, including casing, a filter installed in the casing, and a fan installed in the casing. The casing is defined by an air inlet and an air outlet, and the air outlet is located at the position corresponding to the outlet of the fan. The filter is arranged between the air inlet and the fan, a photocatalytic module is installed in the casing, and the photocatalytic module is arranged between the air inlet and the fan. The wall-mounted air purifier provided in the present application filters and purifies the air by arranging a filter in the casing; and arranging a photocatalytic module in the casing to sterilize and disinfect the gas entering the air inlet and decompose harmful molecules in the gas for better purifying the air and improving the air purification capacity.

### CROSS-REFERENCE TO RELATED APPLICATIONS

The present application claims the priority of the Chinese patent application with application NO. 202122073830.5 filed at the China National Intellectual Property Administration on Aug. 30, 2021, the entire content of which is incorporated into the present application by reference.

### BACKGROUND

### Technical Field

The present application relates to the field of air purification technology, and more particularly, relates to a wall-mounted air purifier.

### Related Prior Arts

As people pay more attention to the environment, more and more air purifiers are used. The current air purifiers are divided into floor-standing and wall-mounted. Floor-standing air purifiers generally have air intake at the bottom or rear side, and air output at the top or front side. The wall-mounted air purifier is generally air-intake at the top and air-out at the front or bottom. The wall-mounted air purifier is generally installed on the wall and will not occupy the floor space of the room, so it is more and more favored by users. However, the current wall-mounted air purifier is mostly installed in the casing to filter the air, which has a weak ability to purify the air.

### SUMMARY

The purpose of the embodiments of the present application is to provide a wall-mounted air purifier to solve the problem of poor air purification ability of the wall-mounted air purifier in the related art.

In order to achieve the above objective, the technical solution adopted in the embodiments of the present application is to provide a wall-mounted air purifier, including a casing, a filter installed in the casing, and a fan installed in the casing. the casing is defined by an air inlet and an air outlet, and the air outlet is located at a position corresponding to the outlet of the fan, and the filter is arranged between the air inlet and the fan, a photocatalytic module is also installed in the casing, and the photocatalytic module is arranged between the air inlet and the fan.

In an optional embodiment, the photocatalytic module includes a photocatalytic net provided in the casing and a light source for illuminating the photocatalytic net, and the light source is installed in the casing.

In an optional embodiment, the photocatalytic module further includes a bracket supporting the photocatalytic net, the bracket is installed in the casing, and the light source is supported by the bracket.

In an optional embodiment, the bracket includes a supporting plate with a U-shaped cross section, an opening is formed between two opposite side walls of the supporting plate, and the light source is arranged at the opening, the photocatalytic net is bent into a U shape, and the photocatalytic net covers the inner surface of the supporting plate, and the supporting plate is defined by a plurality of vents.

In an optional embodiment, the photocatalytic net is provided with holes corresponding to the vent.

In an optional embodiment, the supporting plate is provided with a supporting pillar supporting the light source, and the photocatalytic net is defined by a through hole for the supporting pillar to pass through correspondingly.

In an optional embodiment, the opening of the supporting plate is arranged toward the air inlet.

In an optional embodiment, the light source includes an LED module.

In an optional embodiment, the photocatalytic module is arranged between the air inlet and the filter.

In an optional embodiment, the wall-mounted air purifier further includes a filter net covering the air inlet, and the filter net is detachably installed in the casing.

The beneficial effects of the wall-mounted air purifier provided by the embodiments of the present application are summarized as follows: compared with the related art, the wall-mounted air purifier of the present application filters and purifies the air by arranging a filter in the casing; and a photocatalytic module in the casing to sterilize the gas entering the air inlet, and decompose harmful molecules in the gas, thereby better purifying the air and improving the air purification ability.

### BRIEF DESCRIPTION OF THE DRAWINGS

In order to more clearly describe the technical solutions in the embodiments of the present application, the following will briefly introduce the accompanying drawings used in the description of the embodiments or exemplary technologies. Obviously, the accompanying drawings in the following description are only some embodiments of the present application. For those of ordinary skill in the art can obtain other drawings based on these drawings without creative work.

FIG. 1 is a schematic diagram of a structure a wall-mounted air purifier provided in Embodiment 1 of the present application;

FIG. 2 is a schematic diagram of a structure of the wall-mounted air purifier in FIG. 1 when the filter and filter net are taken out;

FIG. 3 is a schematic diagram of a bottom structure of the wall-mounted air purifier of FIG. 1;

FIG. 4 is a schematic sectional diagram of a structure along the line A-A in FIG. 3;

FIG. 5 is a schematic diagram of a structure of the wall-mounted air purifier in FIG. 1 when the hanging plate is separated;

FIG. 6 is a schematic diagram 1 of an exploded structure of the wall-mounted air purifier of FIG. 1;

FIG. 7 is a schematic diagram 2 of an exploded structure of the wall-mounted air purifier of FIG. 1;

FIG. 8 is a schematic diagram of a structure of the photocatalytic module in the wall-mounted air purifier of FIG. 1;

FIG. 9 is a schematic diagram of an exploded structure of the photocatalytic module in FIG. 8;

FIG. 10 is a schematic cross-sectional diagram of a structure the wall-mounted air purifier provided in the Embodiment 2 of the present application;

FIG. 11 is a schematic diagram of an enlarged structure of the transmitting head in FIG. 10;

FIG. 12 is a schematic cross-sectional structure diagram of the wall-mounted air purifier provided in the Embodiment 3 of the present application;

FIG. 13 is a schematic diagram of a structure of a wall-mounted air purifier provided in Embodiment 4 of the present application;

FIG. 14 is a schematic cross-sectional diagram of a structure of a wall-mounted air purifier provided in Embodiment 5 of the present application.

The main reference numbers of the drawings in the figures are as follows:
100—wall-mounted air purifier;
10—casing; 11—bottom shell; 111—inclined surface; 112—air inlet; 113—baffle; 114—air outlet surface; 115—air outlet; 116—socket; 117—slide plate; 12—housing; 121—wire concealing groove; 122—hanging buckle;
21—filter; 211—magnetic block; 212—magnetic member; 22—partition; 221—chute; 23—filter net; 24—grid plate;
31—fan; 311—wind shield; 312—tubular wind wheel; 313—motor;
40—photocatalytic module; 41—photocatalytic net; 411—through hole; 42—light source; 43—bracket; 431—supporting plate; 4311—hole; 432—supporting pillar;
51—hanging plate; 511—hook; 52—control panel; 53—negative ion generator; 530—transmitting head; 531—negative ion release brush; 532—support; and 533—guiding plate.

### DETAILED DESCRIPTION

In order to make the technical problems to be solved by the present application, technical solutions, and beneficial effects clearer, it further describes the present application in detail with reference to the accompanying drawings and embodiments in the following. It should be understood that the specific embodiments described here are only used to explain the present application, and are not used to limit the present application.

It should be noted that when an element is referred to as being “fixed to” or “arranged on” another element, it can be directly on the other element or indirectly on the other element. When an element is referred to as being “connected to” another element, it can be directly connected to the other element or indirectly connected to the other element.

In addition, the terms “first” and “second” are only used for descriptive purposes, and cannot be understood as indicating or implying relative importance or implicitly indicating the number of indicated technical features. Therefore, the features defined with “first” and “second” may explicitly or implicitly include one or more of these features. In the description of the present application, “a plurality of” means two or more than two, unless otherwise specifically defined. The meaning of “several” is one or more than one, unless otherwise specifically defined.

In the description of The present application, it should be understood that the orientation or positional relationship indicated by the terms of “center”, “length”, “width”, “thickness”, “upper”, “lower”, “front”, “rear”, “left”, ““right”, “vertical”, “horizontal”, “top”, “bottom”, “inner”, “outer”, etc. are based on the orientation or positional relationship shown in the drawings, and are only for convenience of description simplifying the description, rather than indicating or implying that the device or element referred to must have a specific orientation, be constructed and operated in a specific orientation, and therefore should not be construed as a limitation of the application.

In the description of the present application, it should be noted that, unless otherwise clearly specified and limited, the terms of “installation”, “connection” and “connecting” should be understood in a broad sense, for example, it can be a fixed connection or a detachable connection or integrally connected; it can be a mechanical connection or an electrical connection; it can be directly connected or indirectly connected through an intermediate medium, and it can be the internal communication between two components or the interaction between two components. For those of ordinary skill in the art, the specific meanings of the above terms in the present application can be understood according to specific circumstances.

The reference to “one embodiment”, “some embodiments” or “embodiments” described in the specification of the present application means that one or more embodiments of the present application include a specific feature, structure, or characteristic described in combination with the embodiment. Therefore, the sentences of “in one embodiment”, “in some embodiments”, “in some other embodiments”, “in some other embodiments”, etc. appearing in different places in this specification are not necessarily all refer to the same embodiment, but mean “one or more but not all embodiments”, unless otherwise specifically emphasized. In addition, in one or more embodiments, specific features, structures, or characteristics may be combined in any suitable manner.

The English original texts corresponding to the English abbreviations used in the present application are as follows:

LED: Light Emitting Diode;

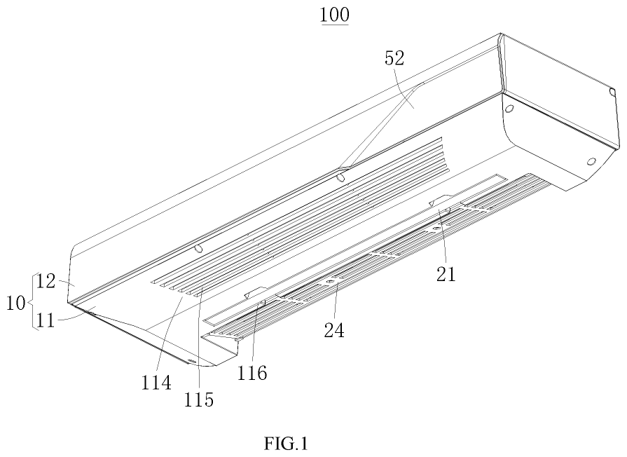

For the convenience of description, refer to FIG. 1 and FIG. 4. The definition in the present application: when the casing 10 is installed on the wall, one side of the casing 10 close to the wall is the rear side of the casing 10 and the wall-mounted air purifier 100, and one side of the casing 10 away from the wall is the front side of the casing 10 and the wall-mounted air purifier 100.

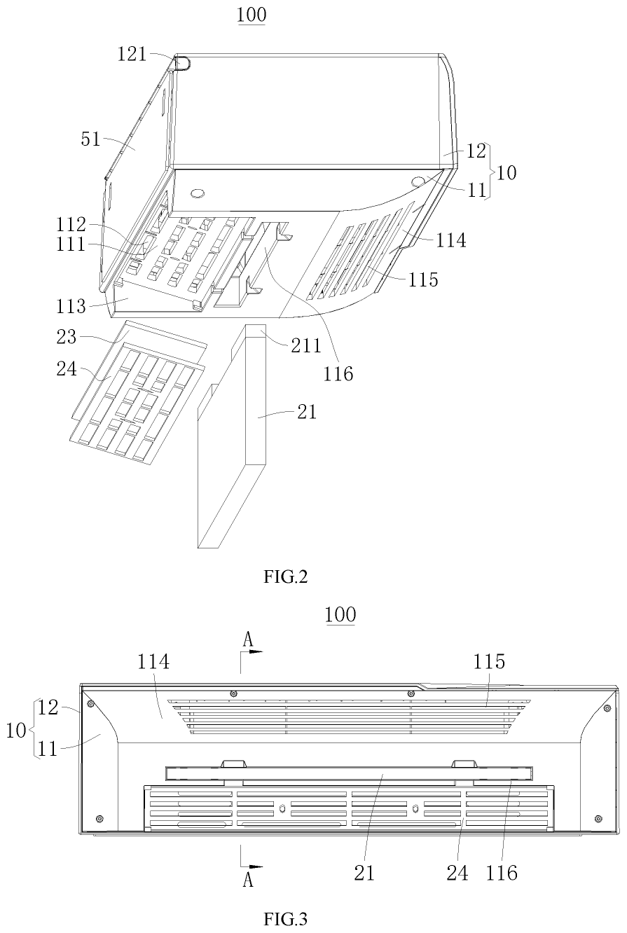

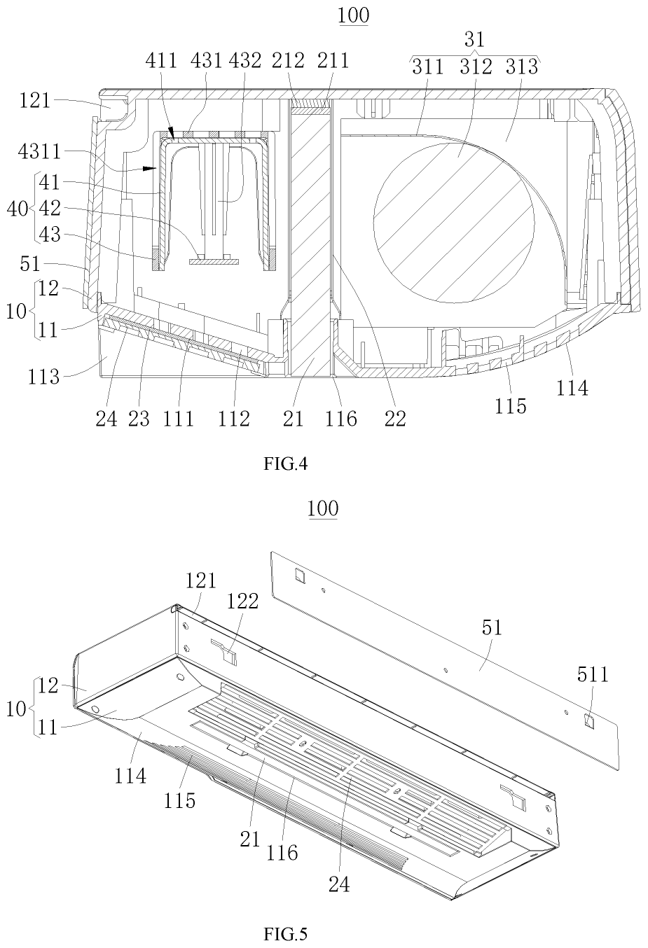

Refer to FIGS. 1-4, FIG. 1 is a schematic diagram of the three-dimensional structure of the wall-mounted air purifier 100 of this embodiment. FIG. 2 is a schematic diagram of the structure when the filter 21 and the filter net 23 of the wall-mounted air purifier 100 of the embodiment are separated from the casing 10. FIG. 3 is a schematic bottom view of the wall-mounted air purifier 100 of this embodiment. FIG. 4 is a schematic cross-sectional diagram of a structure taken along A-A in FIG. 3, and FIG. 4 is a cut-away perspective view of the wall-mounted air purifier 100. FIG. 4 is a schematic sectional view of the structure taken along A-A in FIG. 3.

Referring to FIG. 4, the wall-mounted air purifier 100 provided in the present application will now be described. The wall-mounted air purifier 100 includes: a casing 10, a filter 21, and a fan 31. The filter 21 and the fan 31 are installed in the casing 10, and the casing 10 supports and protects the filter 21 and the fan 31. The casing 10 is provided with an air inlet 112 and an air outlet 115. The air outlet 115 is arranged at a position corresponding to the outlet of the fan 31, and the filter 21 is arranged between the air inlet 112 and the fan 31, so that when the fan 31 works, the air can enter the casing 10 from the air inlet 112, and then be filtered and purified by the filter 21. It is blown out by the fan 31 from the air outlet 115 to purify the air. In addition, the filter 21 is arranged between the air inlet 112 and the fan 31, so that the air will be filtered and purified by the filter 21 before entering the fan 31, thereby reducing the adhesion of impurities on the fan 31.

Refer to FIG. 4, the wall-mounted air purifier 100 also includes a photocatalytic module 40; the photocatalytic module 40 is installed in the casing 10, and the photocatalytic module 40 is supported and protected by the casing 10, and the photocatalytic module 40 is arranged between the air inlet 112 and the fan 31, so that the air is firstly sterilized and disinfected by the photocatalytic module 40 before entering into the fan 31, and the photocatalytic module 40 catalytically decomposes harmful molecules and organic molecules in the airflow to improve the air purification function and promote cleaner air.

Compared with the existing technology, the wall-mounted air purifier 100 provided by the present application is provided with a filter 21 in the casing 10 to filter and purify the air; and a photocatalytic module 40 is installed in the casing 10 to sterilize the gas entering from the air inlet 112 and decompose harmful molecules in the gas to better purify the air and enhance the air purification capacity.

In one embodiment, refer to FIGS. 1 and 4, the air inlet 112 on the casing 10 is located at a corresponding position on the rear side of the bottom of the casing 10. Since the wall-mounted air purifier 100 is generally installed on the wall, the air inlet 112 is arranged at the bottom of the casing 10, so that the air under the wall-mounted air purifier 100 can be directly sucked and purified, so as to more quickly purify the air in the user's active area, thus improving the purified air for users faster and better. Arranging the air inlet 112 at the corresponding position on the rear side of the bottom of the casing 10, the wall can guide and gather the air entering the air inlet 112, so that the air below the casing 10 circulates upwards into the casing 10, which improves the air purification capability and better prevent impurities from falling into the air inlet 112. Of course, in an embodiment, the air inlet 112 may also be arranged on the top surface of the casing 10.

In one embodiment, referring to FIGS. 2 and 4, the bottom of the casing 10 has an inclined surface 111, the air inlet 112 is provided on the inclined surface 111, and the inclined surface 111 extends backward and upward from the bottom of the casing 10 obliquely. The arrangement of the inclined surface 111 and the air inlet 112 on the inclined surface 111. When the casing 10 is installed on the wall, the inclined surface 111 and the wall can form a trumpet-like shape, that is, a trough with an opening on the lower side and gathering on the upper side. Such structure can better gather air and facilitate the gas to enter the air inlet 112 faster, so that the air under the casing 10 can circulate upwards into the casing 10, which improves the air purification capacity.

In one embodiment, the inclined surface 111 is flat to facilitate processing. It is understandable that the inclined surface 111 can also be arranged to be arcuate or curved.

In one embodiment, the bottom of the casing 10 is provided with baffles 113 protruding downwards at both ends of the inclined surface 111, so that it can better cooperate with the wall to form a cavity with a reduced area from bottom to top to better gather air. It is convenient for the gas to enter the air inlet 112 faster, so that the air under the casing 10 circulates upwards into the casing 10, and the air purification capacity is improved.

In an embodiment, referring to FIG. 4, the filter 21 may be a primary filter, a high-efficiency filter, an activated carbon filter, and so on.

In an embodiment, referring to FIGS. 2 and 4, the wall-mounted air purifier 100 also includes a filter net 23, which can be detachably installed on the casing 10, and the filter net 23 covers the air inlet 112 to filter the air through the filter net 23 to reduce impurities such as dust, hair from entering the casing 10. The filter net 23 can be detachably installed on casing 10 for easy removal, cleaning or replacement.

In an embodiment, the wall-mounted air purifier 100 further includes a grille plate 24, which is detachably mounted on the casing 10, and the grille plate 24 is used to support the filter net 23. The grid plate 24 is provided to support the filter net 23 so as to facilitate the installation of the filter net 23 on the casing 10.

In one embodiment, the grid plate 24 can be fixed to the casing 10 by screws to facilitate disassembly. Of course, the grid plate 24 can also be clipped to the casing 10.

In one embodiment, the filter net 23 and the grid plate 24 can be made as a whole, that is, the filter net 23 and the grid plate 24 are an integral structure to support the filter net 23 through the grid plate 24, so that the filter net 23 can be installed in the casing 10 superior. Of course, in some embodiments, the filter net 23 can be arranged separately, and the filter net 23 can be directly fixed on the casing 10. For example, the four sides or the surrounding positions of the filter net 23 can be fixed on the casing 10 by screws, magnetic attraction, buckles and other structures. This structure can reduce the cost, and it is convenient to disassemble the filter net 23 and clean.

In one embodiment, the air outlet 115 on the casing 10 is arranged at the corresponding position on the front side of the bottom of the casing 10, which can blow the air in the area below the casing 10 to better flow, so as to better purify the air, and can also make users under the wall mounted air purifier 100 can breathe the purified air faster, enhancing the user experience.

In one embodiment, the bottom of the casing 10 has an air outlet surface 114, and the air outlet 115 is arranged on the air outlet surface 114. The air outlet surface 114 extends forward and upward from the bottom of the casing 10, so that the air blown out by the air outlet 115 is directed toward the lower front side of the wall-mounted air purifier 100 can better drive the air flow under the wall-mounted air purifier 100, and the purified air can directly cover a larger area, so that the user can breathe the purified air faster and improve the user experience.

In one embodiment, the air outlet surface 114 is arcuate to reduce the edges and corners on the front side of the bottom of the casing 10, improve safety and aesthetics, and also enable the air blown by the air outlet 115 to cover a larger area. Of course, the air outlet surface 114 can also be inclined to facilitate processing.

In one embodiment, the casing 10 includes a bottom shell 11 and a cover 12 covering the bottom shell 11 to facilitate processing. Of course, in other embodiments, the casing 10 can also be formed by combining a plurality of plates.

In one embodiment, when the air inlet 112 is arranged at the bottom of the casing 10, the corresponding air inlet 112 is arranged on the bottom shell 11. Similarly, when the air outlet 115 is arranged at the bottom of the casing 10, the air outlet 115 can be arranged on the bottom shell 11. Of course, when the air inlet 112 is arranged on the top of the casing 10, the corresponding air inlet 112 is arranged on the casing 12.

In one embodiment, referring to FIG. 1, a control panel 52 is arranged on the front side of the casing 10, and the control panel 52 is inclined from top to bottom to the rear side of the casing 10 to facilitate the user's operation and the user to determine the control panel 52. s position. In one embodiment, the control panel 52 is arranged on the casing 12 to facilitate processing and manufacturing.

In one embodiment, referring to FIG. 5. FIG. 5 is a schematic diagram of a structure of the wall-mounted air purifier 100 when the upper hanging plate 51 is separated from the casing 10 in this embodiment. A hanging buckle 122 is arranged on the rear side of the casing 10, so that the casing 10 can be installed on the wall by way of hanging, which is convenient for installation and fixation. Of course, in some other embodiments, the casing 10 can also be installed on the wall using screws.

In one embodiment, referring to FIG. 5, the wall-mounted air purifier 100 further includes a hanging plate 51. The hanging plate 51 is provided with a hook 511 that cooperates with the hanging buckle 122 and is used to fix the hanging plate 51 on the wall. When the hanging plate 51 is arranged, the hanging plate 51 can be fixed on the wall first, and then the casing 10 can be hung on the hanging plate 51, which is convenient for positioning and installation. The hanging plate 51 can be pasted on the wall or fixed on the wall by nails.

In one embodiment, referring to FIG. 5, a wire concealing groove 121 is arranged on the rear side of the casing 10 so that the power supply can be placed in the wire concealing groove 121 to facilitate wiring.

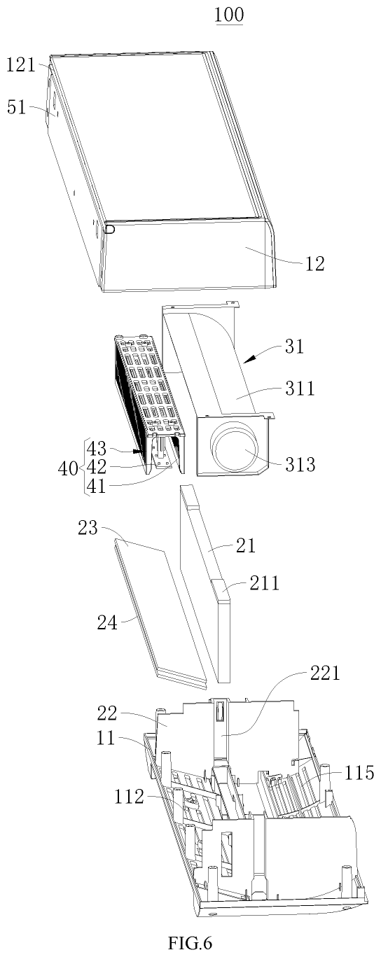

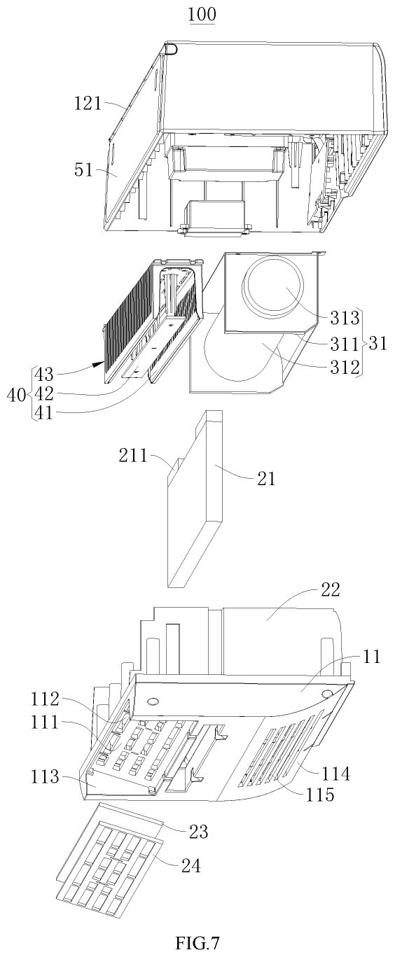

Referring to FIG. 6 and FIG. 7 together. FIG. 6 is schematic diagram of an exploded structure the wall-mounted air purifier 100 of this embodiment at an angle. FIG. 7 is schematic diagram of an exploded structure of the wall-mounted air purifier 100 of this embodiment from another angle.

In one embodiment, referring to FIG. 2, FIG. 6 and FIG. 7. The casing 10 is provided with a socket 116, which is used for plugging and unplugging the filter 21, and the filter 21 can be detachably inserted into casing 10 through socket 116, that is, filter 21 can be is inserted into the casing 10 from the socket 116 to facilitate the installation of the filter 21; of course, the filter 21 can also be extracted from the socket 116 to clean or replace the filter 21. Of course, in other embodiments, the casing 10 can also be disassembled to disassemble the filter 21.

In one embodiment, the socket 116 is arranged on the bottom surface of the casing 10. Since the wall-mounted air purifier 100 is fixed on the wall during use, a socket 116 is arranged on the bottom surface of the casing 10 to facilitate the insertion and removal of the filter 21 from the socket 116, which facilitates the disassembly and replacement of the filter 21. Of course, in other embodiments, a socket 116 may be arranged on the side of the casing 10, and the filter 21 can be inserted and removed from the side of the casing 10.

In one embodiment, the filter 21 is vertically arranged in the casing 10 to facilitate the insertion and removal of the filter 21 from the socket 116. In addition, the space occupied by the filter 21 in the front and rear directions of the casing 10 can be reduced, and the width of the casing 10 in the front and rear directions can be reduced.

In one embodiment, a magnetic member 212 is arranged in the casing 10, and a magnetic block 211 is arranged on the filter 21. The magnetic member 212 and the magnetic block 211 are connected with magnetic member so as to magnetically fix the filter 21 in the casing 10 for convenient disassembly and assembly of the filter 21.

In an embodiment, the magnetic member 212 is a magnet, and the magnetic block 211 may be a magnet with magnetic attraction, such as a magnet matched with the magnetic member 212 for magnetic attraction. Of course, the magnetic member 212 may also be a soft magnet such as an iron block. Of course, the magnetic block 211 is a magnet, and the magnetic member 212 may be a magnet with magnetic attraction, such as a magnet matched with the magnetic block 211 for magnetic attraction. It is understandable, the magnetic block 211 may also be a soft magnet such as an iron block.

In one embodiment, a buckle may be arranged in the casing 10 to clamp the filter 21 in the casing 10, and it may also facilitate the disassembly and assembly of the filter 21.

In an embodiment, referring to FIGS. 6 and 7, two partitions 22 are arranged in the casing 10, and the two partitions 22 are respectively located at the two ends of the filter 21, and the partition 22 is provided with a sliding groove 221, so that when disassemble and assemble filter 21, it can play a guiding role, and when filter 21 is installed in casing 10, the filter 21 can be positioned. In addition, the partition 22 is provided to allow all the air entering the casing 10 to pass through the filter 21 to better purify the air. In addition, the arrangement of the partition 22 can also increase the strength of the casing 10.

In one embodiment, the air inlet 112 and the fan 31 are located between the two partitions 22, so that the air entering from the air inlet 112 will be purified and filtered by the filter 21 to ensure the air purification effect.

In one embodiment, referring to FIGS. 4, 6 and 7, the fan 31 includes a wind cover 311, a tubular wind wheel 312, and a motor 313. The motor 313 is connected to the tubular wind wheel 312 to drive the motor 313 to rotate through the tubular wind wheel 312, and the tubular wind wheel 312 is installed in the wind cover 311, and the air flow is guided by the wind cover 311. By using the tubular wind wheel 312, the volume can be made smaller, and the air volume can be large, so that the casing 10 can be made smaller in volume. Of course, in other embodiments, other fan 31 structures can also be used.

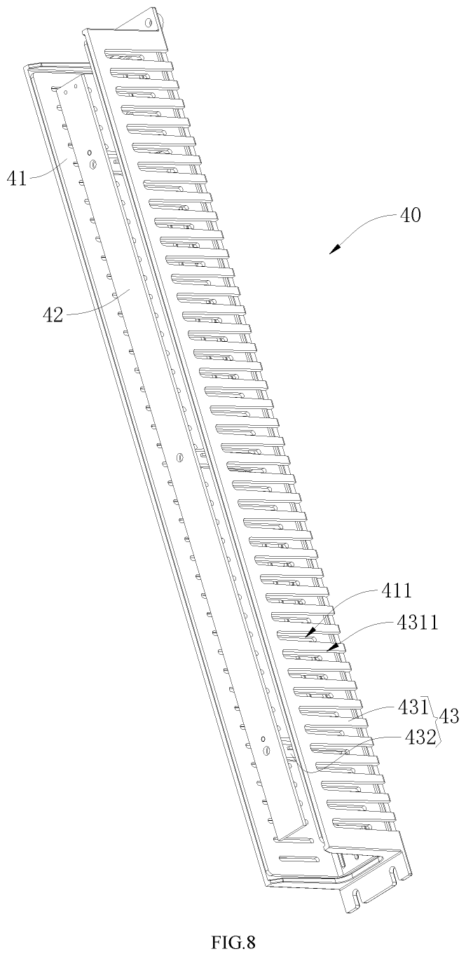

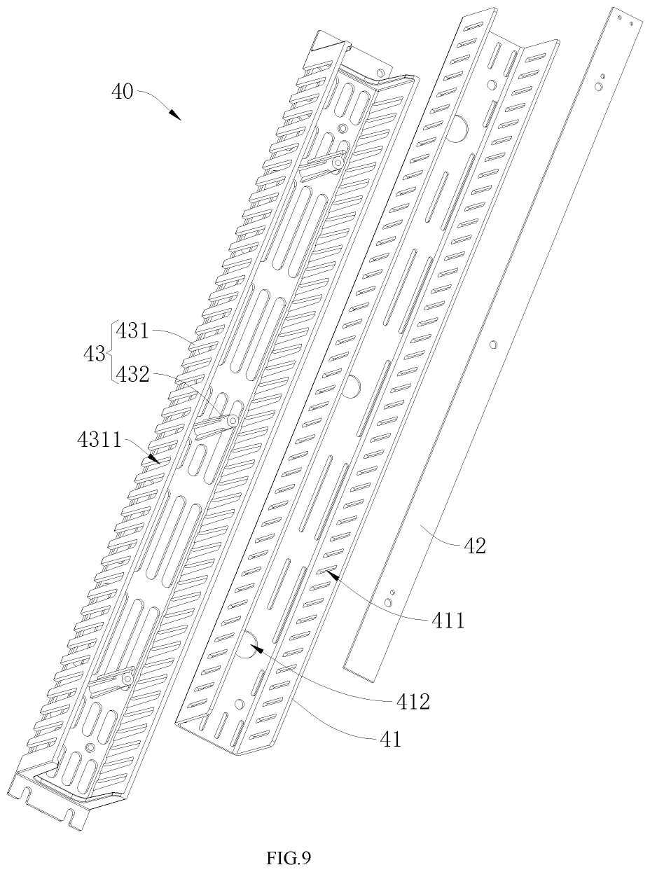

In one embodiment, referring to FIGS. 4, 8, and 9, the photocatalytic module 40 includes a photocatalytic net 41 and a light source 42. Both the photocatalytic net 41 and the light source 42 are installed in the casing 10. The light source 42 is used to illuminate the photocatalytic net 41 so that the airflow passes through the photocatalytic net 41. When the photocatalytic net 41 catalytically decomposes organic molecules and harmful molecules in the airflow, for example, it can decompose formaldehyde, benzene, and TVOC (Total Volatile Organic Compounds), and sterilize, so as to play a role in purification and sterilization, improving purification capacity.

In an embodiment, the photocatalytic module 40 further includes a bracket 43, and the photocatalytic net 41 is installed on the bracket 43, and the photocatalytic net 41 is supported by the bracket 43. The light source 42 is installed on the bracket 43, and the light source 42 is supported by the bracket 43, and it is convenient for the light source 42 to illuminate the photocatalytic net 41, and the photocatalytic module is formed as a whole to facilitate assembly. The bracket 43 is installed in casing 10 to install photocatalytic net 41 and light source 42 in casing 10.

In one embodiment, the bracket 43 includes a supporting plate 431, and the supporting plate 431 is arranged in a U shape. That is to say, the supporting plate 431 is bent into a U shape as a whole, and the cross section of the supporting plate 431 is also in a U shape. An opening can be formed. The photocatalytic net 41 covers the inner surface of the supporting plate 431 so that the photocatalytic net 41 bends into a U-shape, which can facilitate the supporting plate 431 to fix the photocatalytic net 41 and protect the photocatalytic net 41 through the supporting plate 431. The supporting plate 431 is U-shaped. In a smaller space, a larger area of the photocatalytic net 41 can be installed to improve the photocatalytic decomposition ability and improve the purification performance. The light source 42 is arranged at the opening of the supporting plate 431, which can better illuminate the photocatalytic net 41 on the inner surface of the supporting plate 431, which can improve the utilization rate of the light source 42 and save energy. Understandably, the bracket 43 can also be arranged in other shapes as long as it can stably support the photocatalytic net 41. For example, a plurality of frame structures can be arranged to support the photocatalytic net 41, and the plurality of frame structures are combined to form the bracket 43.

In one embodiment, the supporting plate 431 is provided with a plurality of vents, so that the air flow can enter the inside of the supporting plate 431 through the through hole 411, and then pass through the photocatalytic net 41 to be purified by the photocatalytic net 41. In some embodiments, a mesh plate can also be used to support the photocatalytic net 41.

In one embodiment, the photocatalytic net 41 is provided with an opening 4311 corresponding to the vent, which facilitates the air flow through the photocatalytic net 41, reduces the resistance of the air flow through the photocatalytic net 41, and improves the ability of the wall-mounted air purifier 100 to purify air.

In one embodiment, the opening of the supporting plate 431 is arranged toward the air inlet 112, which can facilitate air to enter the photocatalytic net 41 from the opening of the supporting plate 431, and reduce the resistance of the air flow into the photocatalytic net 41.

In one embodiment, the photocatalytic module 40 is arranged between the air inlet 112 and the filter 21, so that the air entering the air inlet 112 can be processed by the photocatalytic module 40 and then filtered by the filter 21. Since the air is catalyzed and decomposed by the photocatalytic module 40, the partial macromolecules in the air are decomposed, which can better avoid clogging the filter 21. Understandably, the photocatalytic module 40 can also be arranged between the filter 21 and the fan 31.

In one embodiment, the photocatalytic module 40 is arranged between the air inlet 112 and the filter 21, and the opening of the supporting plate 431 is arranged toward the air inlet 112. The air entering the air inlet 112 can quickly enter the photocatalytic module 40 for catalytic treatment, and then enter the filter 21 for filtration, and then pass through the fan 31, reducing the resistance of air circulation and improving the ability of air purification.

In one embodiment, the light source 42 includes an LED module, and using the LED module is more energy-efficient. Understandably, the light source 42 can also be other lamps, such as bulbs, light tubes, and so on.

In one embodiment, the LED module includes a substrate and LED lamp beads arranged on the substrate, so as to support the LED lamp beads through the substrate and dissipate the LED lamp beads. In other embodiments, the LED module can also use structures such as LED light bars.

In one embodiment, the supporting pillar 432 is arranged in the supporting plate 431, and the photocatalytic net 41 is correspondingly defined by a through hole 411. During assembly, the supporting pillar 432 passes through the through hole 411 of the photocatalytic net 41 to install the photocatalytic net 41 on the inner surface of the supporting plate 431. With supporting pillar 432, the light source 42 can be installed on supporting pillar 432 so that the bracket 43 can support light source 42. Understandably, the light source 42 can also be directly fixed in the casing 10.

In one embodiment, the photocatalytic module 40 is arranged between the air inlet 112 and the filter 21, so that the air will be catalyzed and decomposed and disinfected by the photocatalytic module 40 before entering the filter 21 to better kill bacteria in the air, decompose harmful molecules, and avoid bacteria and other organisms grow in the filter 21 to protect the filter 21 to a certain extent.

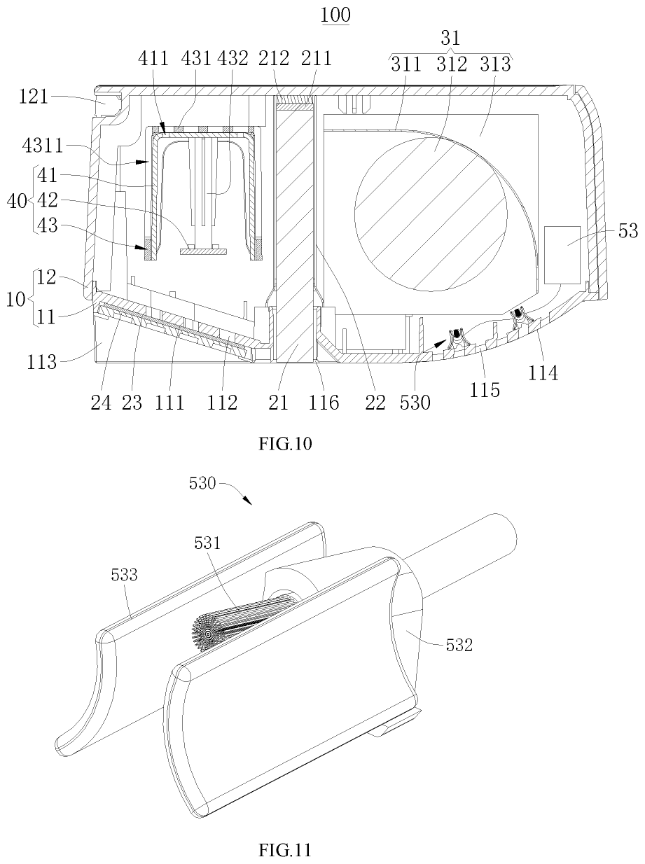

In an embodiment, referring to FIG. 10 and FIG. 11. FIG. 10 is a schematic cross-sectional diagram of a structure of the wall-mounted air purifier 100 of this embodiment. FIG. 11 is a schematic diagram of the structure of the transmitting head of this embodiment. In this embodiment, the wall-mounted air purifier 100 further includes a negative ion generator 53, which has an transmitting head 530, and the transmitting head 530 of the negative ion generator 53 is arranged at the corresponding position of the air outlet 115, so that the air flow generated by the fan 31 passes through the negative ion after the transmitting head 530 of the negative ion generator 53 flows out of the air outlet 115 to form an air current with negative ions, so as to better disinfect and purify the air.

In one embodiment, the transmitting head 530 includes a negative ion releasing brush 531, a support 532, and two guiding plates 533. The support 532 is installed on the casing 10, the negative ion release brush 531 is installed on the support 532, the negative ion release brush 531 is supported by the support 532, and the negative ion release brush 531 is extended to the air outlet 115, and the two guiding plates 533 are respectively arranged on the opposite sides of the negative ion release brush 531, the distance between the two guiding plates 533 gradually expands downward from the middle of the guiding plate 533, that is, the distance between the two guiding plates 533 is arranged by the middle of the guiding plate 533. The direction to the lower side of the guiding plate 533 is gradually expanded, so that the space between the lower parts of the two guiding plates 533 gradually increases, and the airflow flows to the lower part of the two guiding plates 533, which will decelerate and diffuse, thereby making the negative ions in the airflow diffuse better to increase the area covered by the negative ions.

In one embodiment, the guiding plate 533 is fixedly connected to the support 532 to facilitate the installation and fixation of the guiding plate 533, and further facilitate the installation and fixation of the transmitting head 530. Of course, in some embodiments, the guiding plate 533 may also be separately supported in the casing 10.

In one embodiment, the distance between the two guiding plates 533 is gradually expanded upward from the middle of the guiding plate 533, that is, the distance between the two guiding plates 533 is from the middle of the guiding plate 533 to the upper side of the guiding plate 533 is gradually expanded, so that more air enters between the two guiding plates 533, and more air contacts the negative ion release brush 531, and more negative ions are generated.

In one embodiment, the distance between the upper sides of the two guiding plates 533 is smaller than the distance between the lower sides of the two guiding plates 533, so as to ensure that the airflow velocity from the lower sides of the two guiding plates 533 is less than the speed of the external airflow of the two outer guiding plates 533, which will form a certain siphon effect on the underside of the two guiding plates 533 to better diffuse the negative ions in the airflow flowing out between the two guiding plates 533, thereby increasing the coverage area of the negative ions.

In one embodiment, the negative ion release brush 531 is located at a corresponding position above the middle of the guiding plate 533, so that more airflow passes through the negative ion release brush 531, and then is guided and diffused by the guiding plate 533.

In one embodiment, the negative ion release brush 531 extends obliquely upward, that is, the negative ion release brush 531 extends obliquely upward from the support 532 to the free end of the negative ion release brush 531. Since the free end of the negative ion release brush 531 is relatively more open, the negative ion release brush 531 is extended obliquely upwards, which can reduce the resistance of the negative ion release brush 531 to the air flow, thereby facilitating the air flow through the negative ion releasing brush 531 to produce more negative ions.

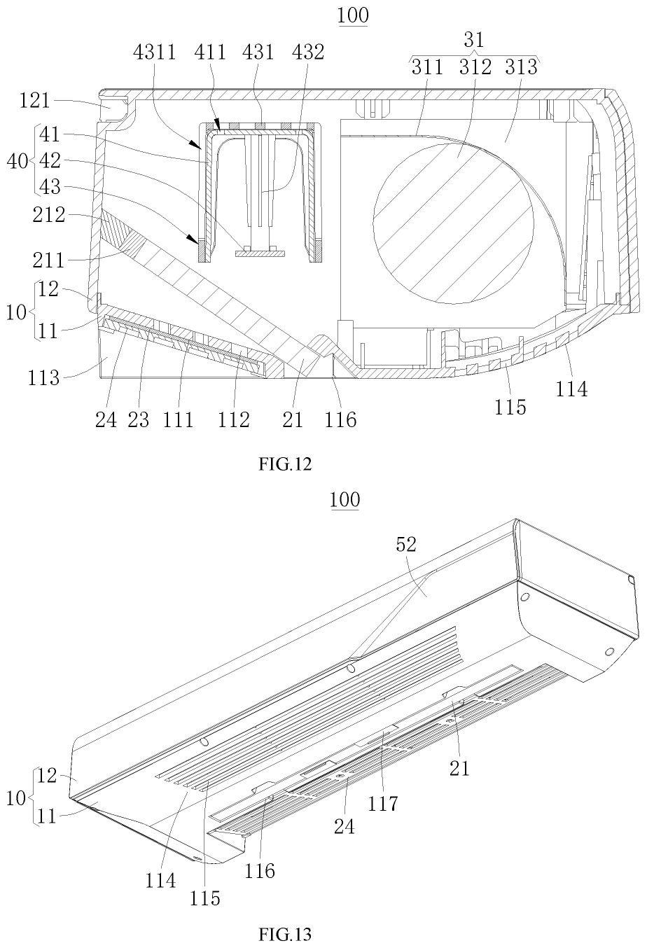

In an embodiment, referring to FIG. 12, which is a schematic cross-sectional diagram of a structure of the wall-mounted air purifier 100 of this embodiment. In this embodiment, the photocatalytic module 40 is arranged between the fan 31 and the filter 21, and the air filtered and purified by the filter 21 is catalytically decomposed and sterilized by the photocatalytic module 40 to ensure the purification effect of the air.

In one embodiment, referring to FIG. 13, which is a schematic diagram of the three-dimensional structure of the wall-mounted air purifier 100 of this embodiment. In this embodiment, the socket 116 is located at the bottom of the casing 10, and the socket 116 is provided with a sliding plate 117, and the sliding plate 117 is slidably installed at the bottom of the casing 10. When the filter 21 is inserted into the casing 10 through the socket 116, the sliding plate 117 can be pulled out to make the sliding plate 117 support the filter 21; when disassembling the filter 21, slide the sliding plate 117 so that the sliding plate 117 is stored in the casing 10, so as to disassemble the filter 21.

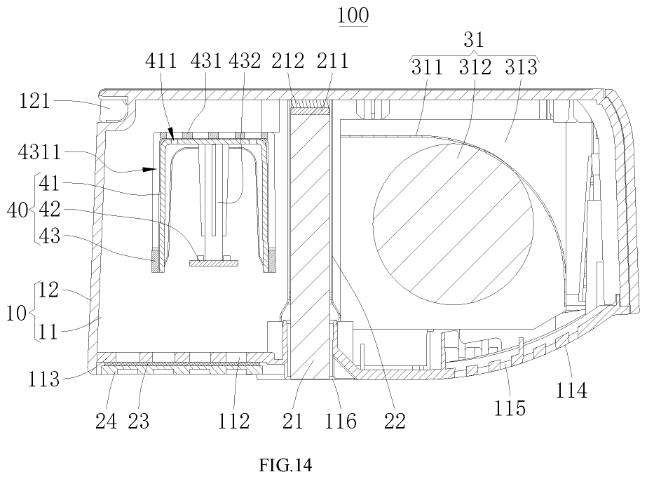

In one embodiment, referring to FIG. 14, which is a schematic cross-sectional diagram of a structure of the wall-mounted air purifier 100 of this embodiment. In this embodiment, the back side of the bottom of the casing 10 is flat, and the air inlet 112 is located on the back side of the bottom of the casing 10. The structure is easy to process and manufacture.

The wall-mounted air purifier 100 of the embodiment of the present application can not only filter and purify, but also sterilize and purify, and has a large air intake, and can provide purified air to the user's surroundings more quickly.

The above are only optional embodiments of the present application and are not intended to limit the present application. Any modification, equivalent replacement and improvement made within the spirit and principle of the present application shall be included in the protection scope of the present application.

What is claimed is:

1. A wall-mounted air purifier, comprising: a casing, a filter installed in the casing, and a fan installed in the casing;
wherein
the casing is provided with an air inlet and an air outlet, and the air outlet is located at a position corresponding to an outlet of the fan, and the filter is arranged between the air inlet and the fan;
a photocatalytic module is also installed in the casing, and the photocatalytic module is arranged between the air inlet and the fan.

2. The wall-mounted air purifier according to claim 1, wherein
the photocatalytic module comprises: a photocatalytic net provided in the casing, and a light source for illuminating the photocatalytic net; and
the light source is installed in the casing.

3. The wall-mounted air purifier according to claim 2, wherein the photocatalytic module further comprises a bracket supporting the photocatalytic net, the bracket is installed in the casing, and the light source is supported by the bracket.

4. The wall-mounted air purifier according to claim 3, wherein the bracket comprises a supporting plate having a U-shaped cross section; an opening is formed between two opposite side walls of the supporting plate, and the light source is arranged at the opening; the photocatalytic net is bent into a U shape, and the photocatalytic net covers an inner surface of the supporting plate; and a plurality of vents are defined in the supporting plate.

5. The wall-mounted air purifier according to claim 4, wherein the photocatalytic net is provided with holes at positions corresponding to the plurality of vents.

6. The wall-mounted air purifier according to claim 4, wherein the supporting plate is provided with a supporting pillar supporting the light source, and the photocatalytic net is corresponding provided therein with a through hole for the supporting pillar to pass through.

7. The wall-mounted air purifier according to claim 4, wherein the opening of the supporting plate is aligned with the air inlet.

8. The wall-mounted air purifier according to claim 2, wherein the light source comprises an LED module.

9. The wall-mounted air purifier according to claim 1, wherein the photocatalytic module is arranged between the air inlet and the filter.

10. The wall-mounted air purifier according to claim 1, further comprising a filter net covering the air inlet, wherein the filter net is detachably installed in the casing.
